SEQUENCE LISTING (9 sequences)

SEQ ID NO: 1 - DNA, 644 bp, homo sapiens
  misc_feature: Mutated GLIS3 patient WRS19
atgggtgtgctttcagtttttgcttgcaatggtgttatgcaagagtaacccaaataacacaagataatgcttaacctagcgaaggaggcaggacctgttcaaacttgctctattcacagatggggtggggtgggcagcaccctcttgtcactgtgacatgtgagctaagaaaatgtatcccaattgctggcttttgagcctttctggaattagatgccattttgtaaaaaaaagtaaatctccttctttgccatgtgaactaaggtgtaaaacggagaaaatgtgttttcaggatttaaaggacctaaagaggcgggtggatcacttgaggtcaggagttcaagaccagcctagccaacatggtgaaactccgtctctactaaaaatacaaaaattagccgggcgcggtggcgggcgcctgtagtcccaggtactcgggaggctgaggcaggagaatggcgtgaacccgggaggcggagcttgcagtgagccgagatcacgccaccgcactccagcctgggcaacagagccagactctgtctcaaaaaaaaaaaaaaaaaaaaaatagagagaatgaataggacctagtatttgatgtcataacagggggattatagtcaataataatttaattgtacatctta

SEQ ID NO: 2 - DNA, 423 bp, homo sapiens
  misc_feature: Flanking region of the deletion in patient WRS25
ccattggccagacccaactggaagctggagggcaaggcagcccagggacatgttccacacaaaccagcctttcagggaagagagcgaggtggaaaatggaaggacagtcagaatacatatctggcgcagtgtgtcaaagttaaattggcagagttttcttcttttttttaaattttattttattattattatactttaagttttagggtacatgtgcacaatgcaggttagttacatatgtatacatgtaccctaaaacttaaagtataataataaaagaaaaaaaaatttttaatccaaaaaattttaatataaaatttaatcccagtagttgttgtttcacggagagattatcacccaggctaacgtacaactgcatgatcatagctcactgcaaccttgaactcttggactcaagcaatc

SEQ ID NO: 3 - protein, 775 aa, homo sapiens
  misc_feature: GLIS3 wild
MMVQRLGLISPPASQVSTACNQISPSLQRAMNAANLNIPPSDTRSLISRESLASTTLSLTESQSASSMKQEWSQGYRALPSLSNHGSQNGLDLGDLLSLPPGTSMSSNSVSNSLPSYLFGTESSHSPYPSPRHSSTRSHSARSKKRALSLSPLSDGIGIDFNTIIRTSPTSLVAYINGSRASPANLSPQPEVYGHFLGVRGSCIPQPRPVPGSQKGVLVAPGGLALPAYGEDGALEHERMQQLEHGGLQPGLVNHMVVQHGLPGPDSQPAGLFKTERLEEFPGSTVDLPPAPPLPPLPPPQGPPPPYHAHAHLHHPELGPHAQQLALPQATLDDDGEMDGIGGKHCYRWIDCSALYDQQEELVRHIEKVHIDQRKGEDFTCFWAGCPRRYKPFNARYKLLIHMRVHSGEKPNKCTFEGCEKAFSRLENLKIHLRSHTGEKPYLCQHPGCQKAFSNSSDRAKHQRTHLDTKPYACQIPGCTKRYTDPSSLRKHVKAHSSKEQQARKKLRSSTELHPDLLTDCLTVQSLQPATSPRDAAAEGTVGRSPGPGPDLYSAPIFSSNYSSRSGTAAGAVPPPHPVSHPSPGHNVQGSPHNPSSQLPPLTAVDAGAERFAPSAPSPHHISPRRVPAPSSILQRTQPPYTQQPSGSHLKSYQPETNSSFQPNGIHVHGFYGQLQKFCPPHYPDSQRIVPPVSSCSVVPSFEDCLVPTSMGQASFDVFHRAFSTHSGITVYDLPSSSSSLFGESLRSGAEDATFLQISTVDRCPSQLSSVYTEG

SEQ ID NO: 4 - protein, 702 aa, homo sapiens
  misc_feature: Mutated GLIS3 625 fs 703 STOP
MMVQRLGLISPPASQVSTACNQISPSLQRAMNAANLNIPPSDTRSLISRESLASTTLSLTESQSASSMKQEWSQGYRALPSLSNHGSQNGLDLGDLLSLPPGTSMSSNSVSNSLPSYLFGTESSHSPYPSPRHSSTRSHSARSKKRALSLSPLSDGIGIDFNTIIRTSPTSLVAYINGSRASPANLSPQPEVYGHFLGVRGSCIPQPRPVPGSQKGVLVAPGGLALPAYGEDGALEHERMQQLEHGGLQPGLVNHMVVQHGLPGPDSQPAGLFKTERLEEFPGSTVDLPPAPPLPPLPPPQGPPPPYHAHAHLHHPELGPHAQQLALPQATLDDDGEMDGIGGKHCYRWIDCSALYDQQEELVRHIEKVHIDQRKGEDFTCFWAGCPRRYKPFNARYKLLIHMRVHSGEKPNKCTFEGCEKAFSRLENLKIHLRSHTGEKPYLCQHPGCQKAFSNSSDRAKHQRTHLDTKPYACQIPGCTKRYTDPSSLRKHVKAHSSKEQQARKKLRSSTELHPDLLTDCLTVQSLQPATSPRDAAAEGTVGRSPGPGPDLYSAPIFSSNYSSRSGTAAGAVPPPHPVSHPSPGHNVQGSPHNPSSQLPPLTAVDAGAERFAPSAPSPHHISPPESSSSFFNTAKNTASLYPAAIRFTPEVLSARNKLFFSTKWYPCPWILWAAAEVLSPTLPRFPENCAACQLLQCGAFV

SEQ ID NO: 5 - DNA, 48 bp, homo sapiens
  misc_feature: Mutated GLIS3 patient WRS19 - 5′ junction
gaaaatgtgttttcaggatttaaaggacctaaagaggcgggtggatca

SEQ ID NO: 6 - DNA, 48 bp, homo sapiens
  misc_feature: Mutated GLIS3 patient WRS19 - 3′ junction
aaactccgtctctactaaaaatacaaaaattagccgggcgcggtggcg

SEQ ID NO: 7 - DNA, 78 bp, homo sapiens
  misc_feature: Mutated GLIS3 patient WRS25 regions framing the deletion
gtacatgtgcacaatgcaggttagttacatatgtatacatgtaccctaaaacttaaagtataataataaaagaaaaaa

SEQ ID NO: 8 - DNA, 3134 bp, homo sapiens
  misc_feature: Mutated GLIS3 cDNA Human
atcctttatggttgaagggtgttggaggaagcagagttttcatcagctgctgaagtggcaactaatagaggtttgaatggccgtagaaaaatattgtgttacttgtttgttgtctaggggctcttggctttgggcctcagtgcaagtccattggaaaaggcagctgcaacaatctagtggtcaccagcagtcccatgatggttcagcgactgggactcatttcacctccagcaagccaggtctctacagcatgcaaccagatcagtcctagcttacagagggcaatgaatgcagccaacctgaatatacctccttcagataccaggtcccttatttcgcgtgagtctttggcgtccacgaccttgagtctgacggaaagtcagtcggcctcaagcatgaagcaggagtggtcccagggctacagggccctcccttcgctctccaaccacggctctcagaatggccttgatctaggggatctccttagccttcctcccgggacatccatgtccagcaatagtgtctctaactcattaccatcctacctttttggcacggaaagtagccactctccttaccctagtcctcggcactcatccaccaggtcccactcggcccgctccaagaagagagcgctgtccttgtccccgctgtccgatggcatcgggatagatttcaataccatcatccgcacgtcgcccacgtccttggtggcctacatcaacgggtcgagggcttcgccggccaacctgtccccgcagccggaggtctacgggcatttcctgggcgtgcgcggcagctgcattccccagccgcgcccggtgcccggcagccagaagggcgtgctggtggcccctggaggcctggcgctgccggcctacggcgaggacggggccctggagcacgagcgcatgcaacagctggagcacggcggcctgcagccaggcctggtcaaccacatggtggtgcagcatggcctgccgggccccgacagccagccggccggcctgttcaagaccgaacgcctggaggagttcccgggcagcaccgtagacctaccccccgcgcctccgctccctcctctgccgccgccccaaggccccccacccccttaccatgcccatgcgcaccttcaccacccggagctcgggccccacgcccagcagctggccttgccccaggccaccctggacgacgacggggagatggacggcatcgggggcaagcattgctaccgctggatcgactgcagcgccctgtacgaccagcaggaggagctcgtgcggcacatcgagaaggtccacatcgaccagcgcaaaggggaggacttcacttgcttctgggccggttgccctcgaagatacaagcccttcaacgcccgctataaactgctgatccacatgagagtccactctggggagaagcccaacaagtgtacgtttgaaggttgcgagaaggccttttcaaggcttgaaaatctcaagatccacttgcggagccacacaggcgagaagccgtatttgtgccagcatccgggttgtcagaaggccttcagtaactccagtgaccgcgccaaacaccagcggacgcatctggacaccaaaccttatgcttgtcaaattccaggatgtaccaaacgctacacagacccaagttccctaagaaagcatgtgaaggcacattcttccaaagagcaacaagcaaggaaaaagttgcggtccagcacagagctccatccagacctgctcacagattgcctcaccgtgcagtccctgcagccggccacttcccctagagatgctgctgctgaagggaccgtgggacgctcccctggacccgggcctgacctctattcagctcccattttctccagcaattattcaagccgaagtggaacagctgctggggccgtaccacccccacatcctgtcagtcacccttctccaggacataatgtacaggggagccctcacaacccctcctcccagttacctccactcacagctgtggacgcaggagctgagaggtttgcaccttctgctccatctcctcaccacatcagccccccggagagttccagctccttcttcaatactgcaaagaacacagcctccctatacccagcagccatcaggttcacacctgaagtcctatcagccagaaacaaactcttcttttcaaccaaatggtatccatgtccatggattttatgggcagctgcagaagttctgtcccccacactaccccgattcccagagaattgtgccgcctgtcagctcctgcagtgtggtgccttcgtttgaggactgcctagtccctacatccatgggccaggccagttttgatgttttccacagagccttctcgactcactcgggcattacagtgtatgatttaccttcaagttcctcgagcctctttggggagtctctccgcagcggggctgaagatgctaccttcttgcagatcagcaccgtggaccgctgtcctagccagctctcctctgtctacaccgaaggctaaaagctctcttggccactcctgcacacccaggaccttgatgttgcttgccaccttttgttttcgtactctcacagactgcttgaggaaggatgtcagcccaatcagcgggccctgcagagttactgatgttctttcagaaggcaggactgaatggcagagctagctttgcaggaatcattacagttgctcctttttctctctggttttcctggactatgtgaccaggaggactgtcttttcaaagggaatttagagcctcactctcctggatacctgttacttcctggctgtcaacccttaaaggaccccagtgaaatgatgtgcatatgaaaatgtagtttggggatgactctacctgaaaactccaaacagggaaaaagacctcaattacgagaggcatagtgacctgcctcacctaatcctcacccttgctgctcctgatgcaaaaccaggcacaacaagggaaatccatgtccaaagcatagtgtggtctcaactcttagaaactccctgggtttcccttttctcttagccacatgctaaccagttgcttctcagtgtttctcttcaatagaaaaataaggttttatatgccaaaaaaaaaaaaaaaa

SEQ ID NO: 9 - DNA, 73 bp, homo sapiens
  misc_feature: Region of mutated GLIS3 cDNA Human around position 2068
caccttctgctccatctcctcaccacatcagccccccggagagttccagctccttcttcaatactgcaaagaa

(19) United States
(12) Patent Application Publication
(10) Pub. No.: US 2011/0112015 A1
(43) Pub. Date: May 12, 2011

(54) USE OF GLIS3 FOR PREPARING FUNCTIONAL PANCREATIC BETA-CELLS

(75) Inventors: Cecile Julier, Paris (FR); Doris Taha, Jeddah (SA); Marc Nicolino, Lyon (FR); Douglas Cavener, State College, PA (US)

(73) Assignee: Consortum National De Recherche En Genomique (CNRG)

(21) Appl. No.: 12/296,878

(22) PCT Filed: Apr. 16, 2007
(86) PCT No.: PCT/IB2007/002165

§ 371 Date: Jan. 29, 2009

Related U.S. Application Data
(60) Provisional application No. 60/791,895, filed on Apr. 14, 2006.

(51) Int. Cl. A61K 38/17; C07H 21/04; C07K 14/47; C12N 5/00; C12N 5/071; A61P 5/14; A61P 3/10; A61P 27/06; C12N 15/63; C12Q 1/68

(52) U.S. Cl. 514/7.3; 536/24.33; 536/24.31; 530/350; 435/377; 435/366; 514/6.9; 514/9.7; 514/20.8; 435/320.1; 435/6

(57) ABSTRACT

The present invention relates to a method of diagnosis of neonatal diabetes, congenital hypothyroidism and congenital glaucoma, comprising detecting the presence of a mutation in the GLIS3 gene and to a method for preparing functional pancreatic beta-cells by culturing human multipotent or pluripotent cells, such as embryonic stem cells (ES-cells) or human somatic stem cells in a culture medium comprising a effective amount of GLIS3 to induce said cells differenciation into functional pancreatic beta-cells producing insulin. It also relates to a pharmaceutical composition comprising GLIS3 as active ingredient.

### FIELD OF THE INVENTION

The present invention relates to a method of diagnosis of neonatal diabetes, congenital hypothyroidism and congenital glaucoma, comprising detecting the presence of a mutation in the GLIS3 gene and to a method for preparing functional pancreatic beta-cells by culturing human multipotent or pluripotent cells, such as embryonic stem cells (ES-cells) or human somatic stem cells in a culture medium comprising a effective amount of GLIS3 to induce said cells differenciation into functional pancreatic beta-cells producing insulin. It also relates to a pharmaceutical composition comprising GLIS3 as active ingredient

### BACKGROUND OF THE INVENTION

Permanent neonatal diabetes may occur either in isolation or associated with multi-organ syndromes. In a consanguineous family from Saudi Arabia, permanent neonatal diabetes was associated with intra-uterine growth retardation, congenital hypothyroidism, facial anomalies, congenital glaucoma, hepatic fibrosis and polycystic kidneys¹. We studied this family and two other consanguineous families affected by a similar syndrome, with notable variations: in the second family (one patient), liver and kidneys were normal, and in the third family (two patients), liver, kidneys and eyes were normal. All patients shared neonatal diabetes, congenital hypothyroidism and a similar facial dysmorphology, leading us to temptatively consider them as affected by the same disease entity, NDH (Neonatal Diabetes and congenital Hypothyroidism).

In connection with the present invention, we showed that mutations in the GLIS3 gene are responsible for the above rare autosomal recessive syndrome characterized by permanent neonatal diabetes, congenital hypothyroidism and other clinical manifestations that are variable between families. We also established the structure of the GLIS3 gene, encoding GLI similar 3, a recently identified transcription factor (Kim et al., Nucleic Acids Res 31:5513-5525, 2003). This gene extends over a long chromosome region (524 kb), contains 32 exons, with multiple 5′ and 3′ ends and alternative exons, which can generate a large variety of transcripts, encoding proteins with extensive variation, especially in their N- and C-terminal ends, and with important variation between tissues.

In the original family, a frameshift mutation was predicted to result in a truncated protein. In the two other families, with an incomplete syndrome, patients harbored deletions affecting the 12 or 11 most 5′ exons of the gene. The absence of a major pancreas and thyroid transcript (both deletions) and an eye-specific transcript (one deletion), together with residual expression of some GLIS3 transcripts, appears to explain the incomplete clinical manifestations in these patients.

We then discovered that GLIS3 is expressed in the pancreas from early development stages (E15.5), when β-cell neogenesis takes place, to adulthood, with increased expression in β-cells compared to other endocrine cells and exocrine tissue (expression ratio: 8:2:1). These results demonstrate a major role for GLIS3 in the development of the pancreatic β-cells, thyroid, eye, liver and kidney.

Therefore, we demonstrate herein that GLIS3 plays a critical role during pancreatic development, and particularly that of β-cells.

Taken together, our findings have important clinical and therapeutic applications, principally of three types:
Diagnosis and treatment of neonatal diabetes, congenital hypothyroidism and congenital glaucoma,
Diagnosis and treatment of frequent diseases associated with the above syndrome: type 1 diabetes, type 2 diabetes, other forms of diabetes, hypothyroidism and glaucoma, and
Applications in cell therapy and regeneration of pancreatic β cells for treating diabetes.

### DESCRIPTION OF THE INVENTION

We showed that mutations in the GLIS3 gene are responsible for a rare autosomal recessive syndrome characterized by permanent neonatal diabetes, congenital hypothyroidism and other clinical manifestations that are variable between families. This study was performed on three independent consanguineous families. One of the families showed the full syndrome, as previously described by Doris Taha (Taha et al., Am J Med Genet 122: 269-273, 2003), characterized by permanent neonatal insulin-dependant diabetes, congenital hypothyroidism, hepatic fibrosis, polycystic kidneys, congenital glaucoma and particular facial dysmorphology. The two other families showed the same pancreatic and thyroid features, but no ocular, renal and hepatic disease (one family), and no renal and hepatic disease (one family).

We established the structure of the GLIS3 gene, encoding GLI similar 3, a recently identified transcription factor (Kim et al., Nucleic Acids Res 31:5513-5525, 2003). This gene extends over a long chromosome region (524 kb), contains 32 exons, with multiple 5′ and 3′ ends and alternative exons, which can generate a large variety of transcripts, encoding proteins with extensive variation, especially in their N- and C-terminal ends, and with important variation between tissues.

In the original family, a frameshift mutation was predicted to result in a truncated protein. In the two other families, with an incomplete syndrome, patients harbored deletions affecting the 12 or 11 most 5′ exons of the gene. The absence of a major pancreas and thyroid transcript (both deletions) and an eye-specific transcript (one deletion), together with residual expression of some GLIS3 transcripts, appears to explain the incomplete clinical manifestations in these patients.

GLIS3 is expressed in the pancreas from early development stages (E15.5), when β-cell neogenesis takes place, to adulthood, with increased expression in β-cells compared to other endocrine cells and exocrine tissue (expression ratio: 8:2:1). These results demonstrate a major role for GLIS3 in the development of the pancreatic β-cells, thyroid, eye, liver and kidney.

### Method of Diagnostic

Thus, the present invention provides a method of diagnosis of neonatal diabetes, congenital hypothyroidism and congenital glaucoma, comprising detecting the presence or the absence of a mutation in the GLIS3 gene selected for stop mutations, deletions and insertions resulting in frame-shift, wherein the presence of said mutation in the sample of a patient or in a prenatal sample is indicative of a neonatal diabetes, congenital hypothyroidism and congenital glaucoma.

The above method may be practiced by any method known in the art including the use of probes after PCR amplification of genomic DNA or RT-PCR of RNA extracted from the sample, gel electrophoresis and northern blot to identify variants of the GLIS3 protein.

For example, in a particular embodiment, the method comprises the use of probes to detect the following deletions and insertions such as in patients WRS19 and WRS25; as well as stop and frame shift mutations in patient WRS15.

### Patients WRS19 (Two Patients):

The A and B sequences correspond to both extremities of the deletion. A sequence is inserted corresponding to sequence I originating from telomeric region of chromosome 9. Position of the deletion on the genomic sequence of reference (NT_{—}008413.16): . . . 4249914[DEL4249915-4398572; substituted by seq [symbol] I [symbol] ]4398573 . . .

size of the deletion: 148658 bp
size of the inserted sequence I: 82 bp

| AL162419.34 (A, flanking, left) | |
|---|---|
| 14401 | atgggtgtgc tttcagtttt tgcttgcaat ggtgttatgc aagagtaacc caaataacac |
| 14461 | aagataatgc ttaacctagc gaaggaggca ggacctgttc aaacttgctc tattcacaga |
| 14521 | tggggtgggg tgggcagcac cctcttgtca ctgtgacatg tgagctaaga aaatgtatcc |
| 14581 | caattgctgg cttttgagcc tttctggaat tagatgccat tttgtaaaaa aaagtaaatc |
| 14641 | tccttctttg ccatgtgaac taaggtgtaa aacggagaaa atgtgttttc aggatttaaa |
| 14701 | ggaccta |
| Seq interstitielle (I): | |
| aagaggcgggtggatcacttgaggtcaggagttcaagaccacctagccaacatggtgaaactccgtactact | |
| aaaaatac | |
| AL162419.34 (B, flanking, right): | |
| | aaaaa ttagccgggc |
| 163381 | gcggtggcgg gcgcctgtag tcccaggtac tcgggaggct gaggcaggag aatggcgtga |
| 163441 | acccgggagg cggagcttgc agtgagccga gatcacgcca ccgcactcca gcctgggcaa |
| 163501 | cagagccaga ctctgtctca aaaaaaaaaa aaaaaaaaaa atagagagaa tgaataggac |
| 163561 | ctagtatttg atgtcataac agggggatta tagtcaataa taatttaatt gtacatctta |

A-I-B forms SEQ ID No 1.

In particular, the invention is directed to the use of primers or probes consisting of from 10 to 30 consecutive nucleotides of:

SEQ ID No 5—mutated GLIS3 patient WRS19-5′ deletion-insertion junction human

gaaaatgtgttttcaggatttaaaggacctaaagaggcgggtggatca

SEQ ID No 6—mutated GLIS3 patient WRS19-3′ deletion-insertion junction human

aaactccgtctctactaaaaatacaaaaattagccgggcgcggtggcg

### Patient WRS25:

Flanking region of the deletion: underligned is the telomeric limit (clone AL133283), in bold centromeric limit (clone AL136231)

(SEQ ID No 2)

ccattggccagacccaactggaagctggagggcaaggcagcccagggaca
tgttccacacaaaccagcctttcagggaagagagcgaggtggaaaatgga
aggacagtcagaatacatatctggcgcagtgtgtcaaagttaaattggca
gagttttcttcttttttttaaattttattttattattattatactttaa
gttttagggtacatgtgcacaatgcaggttagttacatatgtatacatgt
[DEL425700bp]accctaaaacttaaagtataataataaaagaaaa
aaaaatttttaatccaaaaaattttaatataaaatttaatcccagtagtt
gttgtttcacggagagattatcacccaggctaacgtacaactgcatga
tcatagctcactgcaaccttgaactcttggactcaagcaatc

In particular, the invention is directed to the use of primers or probes consisting of from 10 to 30 consecutive nucleotides of SEQ ID No 7 and comprising at least one nucleotide from each part framing the deletion:

gtacatgtgcacaatgcaggttagttacatatgtatacatgt[DEL4
25700bp]accctaaaacttaaagtataataataaaagaaaaaa

Position of the deletion on the contig of reference (NT_{—}008413.16):

### . . . 4176076[DEL:4176077-4601776]4601777 . . .

deletion size: 425700 bp

### Mutation (Family WRS15):

### Nature of the Mutation:

BC033899-insC2067-2068 (cDNA) or AL137071-insC92318-92319 (genomic): Insertion of a [symbol] C [symbol] between nucleotides 2067 and 2068 of the GLIS3 gene, sequence cDNA Genbank: BCO33899, or between nucleotides 92318-92319 (genomic)

This mutation leads to a frameshift beginning at position 625 with an altered sequence till position 702 (see in bold below), and a STOP in position 703 (625FS703STOP):

GLIS3 wild (NP_689842.2, 775 AA) - (SEQ ID No 3):
MMVQRLGLISPPASQVSTACNQISPSLQRAMNAANLNIPPSDTRSLISRESLASTTLSLTESQSASSMKQEWSQG
YRALPSLSNHGSQNGLDLGDLLSLPPGTSMSSNSVSNSLPSYLFGTESSHSPYPSPRHSSTRSHSARSKKRALSL
SPLSDGIGIDFNTIIRTSPTSLVAYINGSRASPANLSPQPEVYGHFLGVRGSCIPQPRPVPGSQKGVLVAPGGLA
LPAYGEDGALEHERMQQLEHGGLQPGLVNHMVVQHGLPGPDSQPAGLFKTERLEEFPGSTVDLPPAPPLPPLPPP
QGPPPPYHAHAHLHHPELGPHAQQLALPQATLDDDGEMDGIGGKHCYRWIDCSALYDQQEELVRHIEKVHIDQRK
GEDFTCFWAGCPRRYKPFNARYKLLIHMRVHSGEKPNKCTFEGCEKAFSRLENLKIHLRSHTGEKPYLCQHPGCQ
KAFSNSSDRAKHQRTHLDTKPYACQIPGCTKRYTDPSSLRKHVKAHSSKEQQARKKLRSSTELHPDLLTDCLTVQ
SLQPATSPRDAAAEGTVGRSPGPGPDLYSAPIFSSNYSSRSGTAAGAVPPPHPVSHPSPGHNVQGSPHNPSSQLP
PLTAVDAGAERFAPSAPSPHHISPRRVPAPSSILQRTQPPYTQQPSGSHLKSYQPETNSSFQPNGIHVHGFYGQL
QKFCPPHYPDSQRIVPPVSSCSVVPSFEDCLVPTSMGQASFDVFHRAFSTHSGITVYDLPSSSSSLFGESLRSGA
EDATFLQISTVDRCPSQLSSVYTEG*
mutated GLIS3: 625 fs 703 STOP (SEQ ID No 4):
MMVQRLGLISPPASQVSTACNQISPSLQRAMNAANLNIPPSDTRSLISRESLASTTLSLTESQSASSMKQEWSQG
YRALPSLSNHGSQNGLDLGDLLSLPPGTSMSSNSVSNSLPSYLFGTESSHSPYPSPRHSSTRSHSARSKKRALSL
SPLSDGIGIDFNTIIRTSPTSLVAYINGSRASPANLSPQPEVYGHFLGVRGSCIPQPRPVPGSQKGVLVAPGGLA
LPAYGEDGALEHERMQQLEHGGLQPGLVNHMVVQHGLPGPDSQPAGLFKTERLEEFPGSTVDLPPAPPLPPLPPP
QGPPPPYHAHAHLHHPELGPHAQQLALPQATLDDDGEMDGIGGKHCYRWIDCSALYDQQEELVRHIEKVHIDQRK
GEDFTCFWAGCPRRYKPFNARYKLLIHMRVHSGEKPNKCTFEGCEKAFSRLENLKIHLRSHTGEKPYLCQHPGCQ
KAFSNSSDRAKHQRTHLDTKPYACQIPGCTKRYTDPSSLRKHVKAHSSKEQQARKKLRSSTELHPDLLTDCLTVQ
SLQPATSPRDAAAEGTVGRSPGPGPDLYSAPIFSSNYSSRSGTAAGAVPPPHPVSHPSPGHNVQGSPHNPSSQLP
PLTAVDAGAERFAPSAPSPHHISPPESSSSFFNTAKNTASLYPAAIRFTPEVLSARNKLFFSTKWYPCPWILWAA
AEVLSPTLPRFPENCAACQLLQCGAFV*

In particular, the invention is directed to the use of primers or probes consisting of from 10 to 30 consecutive nucleotides of SEQ ID No 7 or No 8 and comprising the C insertion at position 2068 or as dipected below:

SEQ ID No 8
caccttctgctccatctcctcaccacatcagcccccc*ggagagttcca
gctccttcttcaatactgcaaagaa

The probes according to the invention may comprise a sequence displaying from 10 to 30, such as 10, 12 15 or 20, consecutive nucleotides of the above sequences SEQ ID No 1, 2, 5, 6 7, 8 and 9 or of a complementary sequence thereof, or of a sequence encoding SEQ ID No 4 especially at position 2068 of the GLIS3 gene to detect the presence of the inserted cytosine. Probes may be labeled with fluorescent or bioluminescent molecules. In addition, the invention provides primers to specifically amplify DNA sequences comprising the above mutated region. In a particular embodiment, the method comprises a RT-PCR reaction on the RNA extracted from the sample using said primers.

The above method may be applied to the prenatal, preclinical and clinical diagnosis of congenital diseases associated with this syndrome including neonatal diabetes, hypothyroidism and glaucoma.

The above method may be extended to the diagnosis, improved understanding and treatment of frequent diseases associated with this syndrome including type 1 diabetes, type 2 diabetes, other forms of diabetes, hypothyroidism and glaucoma. In particular, genetic linkage of the region overlapping with the GLIS3 gene has been previously reported with type 2 diabetes, and with the age at onset of type 2 diabetes (Duggirala et al., Am J Hum Genet 64:1127-1140, 1999), suggesting a possible implication of this gene in the susceptibility to type 2 diabetes. This may have implication for improving the quality of the treatment, by adjusting the nature of the treatment, to the nature of the individual genetic risk variants.

Regarding the possible role of GLIS3 in neonatal diabetes and frequent diabetes, we consider that this gene plays a role in the development and function of pancreatic β-cells: 1) mutations with major functional consequences on GLIS3 function result in defective development/function of β-cells from birth and even before, resulting in neonatal diabetes; 2) Minor genetic variants in this gene, that may exist in the general population, could modulate the number, mass, survival or function of β-cells, hence affecting the individual risk of type 1 or type 2 diabetes later in life.

Thus, in still a further embodiment, the invention relates to the use of GLIS3 protein (SEQ ID No 3) to produce functional pancreatic beta-cells.

The better understanding of the mechanisms involved in the development and function of the organs related to this syndrome, in particular pancreatic β-cells, allows to design strategies to improve the development of these organs (or cells), ex vivo or in vivo, with important therapeutic consequences. Our results showing increased expression of GLIS3 in pancreatic β-cells, compared to other pancreatic tissues, and at early stages of β-cell neogenesis, together with the fact that GLIS3 is a transcription factor, strongly support that GLIS3 plays a critical role in the differentiation, development and function of pancreatic β-cells. One immediate consequence of our discovery is for the design of therapeutic strategies using cell replacement therapy or regenerative therapy for the treatment of diabetes or in cases of pancreatectomised patients. For this, the understanding of pancreatic β-cells differentiation, including the identification of the factors involved in this differentiation, is a critical step. Indeed, this has been recognized by the major funding bodies for diabetes research, which have launched important research programs to encourage research into β-cell development and cell replacement and regenerative therapy for diabetes (e.g. JDRF, NIH and the EU FP6). We provide two main strategies to meet this goal:

### Cell Replacement Therapy for Treating Diabetes

The goal is to replace the deficient pancreatic β-cells from diabetic patients by cells that express insulin. Although there has been encouraging results using cell replacement therapy for diabetes in the past (e.g. Shapiro et al., N Eng J Med 343:230-238, 2000), the success has been partial to date, at least in the case of type 1 diabetes, which is probably the principal indication, with follow-up studies showing that less than 10% of patients remained free of insulin therapy after 5 years following the allo-graft of islets (Ryan et al., Diabetes 54: 2060-2069, 2005). In comparison, better success was obtained in the case of islet transplantation (auto-graft) after pancreatectomy performed for chronic pancreatitis (Robertson et al., Diabetes 50: 47-50, 2001). The main problem in these strategies to date has been to obtain sufficient material, to maintain it in the patient in the case of type 1 diabetes (requiring immunosuppressive treatment) and to keep it producing insulin after transplantation. Hence, alternative strategies to whole islets transplantations has been to differentiate pancreatic β-cellsfrom multipotent or pluripotent cells, such as embryonic stem cells (ES-cells). The process to differentiate ES-cells into pancreatic β-cells requires the identification of the critical factors required, and the sequential order in which they act during the differentiation process. Some of these factors are known, such as PDX1. Recent successes has been obtained using a combination of epidermal growth factor and gastrin for the differentiation of β-cells and their use in replacement therapy in mouse models of diabetes (Suarez-Pinzon et al, Diabetes 54:2596-25601; Rooman and Bouwens, Diabetologia 47: 259-265, 2004). Based on our findings, GLIS3 is a transcription factors that is required for β-cells differentiation, and is thus be a critical determinant for in vitro differentiation from ES-cells.

In this regard, the invention contemplates a method for preparing functional pancreatic beta-cells by culturing human multipotent or pluripotent cells, such as embryonic stem cells (ES-cells) or human somatic stem cells in a culture medium comprising a effective amount of GLIS3 to induce said cells differenciation into functional pancreatic beta-cells producing insulin. It also relates to a culture medium comprising an effective amount of GLIS3 as well as to a cell culture comprising the above cells in a culture media comprising GLIS3. The media according to the invention may further comprise EGF and PDX1.

### Regenerative β-Cells Therapy

As explained above GLIS3 is able to regenerate β-cells from multipotent cells, or to slow down the loss of existing β-cells. Hence, we propose to design strategies to provide the required factors (i.e. GLIS3) in vivo in this aim. This could be achieved by direct administration of the protein to the patients, or using genetic engineering designed vectors that will synthesize this protein.

In this regard, the invention is directed to a method of treatment of diabetes such as type 1 diabetes, type 2 diabetes, other forms of diabetes, hypothyroidism and glaucoma comprising administering an effective amount of GLIS3 to a patient in need of such treatment. It also relates to a pharmaceutical composition comprising GLIS3 as active ingredient. Alternatively, the invention is aimed at a gene therapy vector expressing GLIS3 and at a medicament comprising such vector.

### DESCRIPTION OF THE DRAWINGS

FIG. 1. Linkage mapping and fine mapping in the three NDH families.

a. Genotypes of chromosome 9p markers showing haplotypes segregating in the families. The homozygous regions in red define the interval of linkage in each family. The bracket defines the region of linkage based on the study of the three families. b. Fine mapping using additional microsatellite markers (RPT code) and simple PCR assays (PCR code), identifying homozygous deletions in families NDH2 and NDH3 (shown in black in the chromosome sketches on the right). Details of RPT and PCR markers are provided in Supplementary Table 2a,b on line.

FIG. 2. Characterization of mutation and deletions in NDH patients.

a. Schematic representation of chromosome 9p region showing the location of deletions in NDH2 and NDH3 patients. Black boxes correspond to NCBI RefSeq genes (GLIS3: NM_{—}152629.2, SLC1A1: NM_{—}004170.4), and grey boxes to additional potential exons based on AceView predictions (GLIS3). Open boxes represent the position of deletions observed in NDH2 and NDH3 patients (Del NDH2 and Del NDH3), with the sequences flanking the deletions (bold), and the intervening sequence derived from a region of chromosome 9q (boxed). In NDH3, the 149 kb deleted segment was substituted by 82 bp originating from the telomeric region of chromosome 9q (98% homology). The map position of deletion breakpoints on NT_{—}008413.16 is:

### NDH2: . . . 4176076 [DEL:4176077-4601776] 4601777 . . .

NDH3: . . . 4249914 [DEL:4249915-4398572; substituted by 82 bp (boxed)] 4398573 . . .

b. NDH1 mutation: predicted truncated protein. The sequence shown corresponds to GLIS3 RefSeq (NM_{—}152629.2). Crossed box: zinc finger domains, left hatched box: serine-rich domain, right hatched boxes: proline-rich domain, grey box: Nuclear Localization Signal (NLS), dotted box: frameshifted translation. c. Expression study in NDH2 and NDH3 families. For GLIS3, RT-PCR was performed in family members with single PCR (arrow GLIS3(a)) and nested PCR (arrow GLIS3(b)); For SLC1A1, RT-PCR was performed using nested PCR; a control RT-PCR was performed using primers located in the ubiquitously expressed PCBD1 gene. d. Allele specific GLIS3 expression quantification. GLIS3 alleles were quantified on GLIS3 transcripts amplified from whole blood by nested PCR and on genomic DNA (single PCR) using a StyI RFLP corresponding to the exonic SNP rs6415788, located in GLIS3 RefSeq exon 3. The relative intensity of the 455 bp band (C-allele, corresponding to the “deleted” allele) and 365 bp (A-allele, corresponding to the normal allele) was measured in control individuals and in heterozygous parents (NDH3-1 and 2). The ratios of the C to A alleles, after adjusting for the fragment length, is shown. The genotype of the same controls and of the complete NDH3 family was determined on genomic DNA by PCR using different primers and Styl digestion (C allele: 567 bp, A allele: 387 bp). The experiment was repeated once, with similar results. Primer sequences are provided in Supplementary Table 2d,e on line.

FIG. 3. Human GLIS3 gene expression study in multiple tissues Northern blots

Human multiple tissues Northern blot hybridization with a GLIS3 probe. A 275 bp cDNA probe was generated on kidney cDNA using primers located in GLIS3 RefSeq exons 6 and 7, encompassing amino acids 513-604, and hybridized simultaneously to the two Northern blots. Primer sequences used to generate this probe are provided in Supplementary Table 2h on line. Tissues are: 1: heart, 2: brain, 3: placenta, 4: lung, 5: liver, 6: skeletal muscle, 7: kidney, 8: pancreas, 9: stomach, 10: thyroid, 11: spinal cord, 12: lymph node, 13: trachea, 14: adrenal gland, 15: bone marrow. Other DNA probes covering different regions of the gene were also used in Northern blot hybridization, to help define the gene structure (not shown).

FIG. 4. Glis3 expression studies in mouse pancreas and other tissues, pancreas subfractions and during mouse development

Glis3 expression was quantified by real-time quantitative PCR in a) four tissues at ages 22 days (open boxes) and 35 days (black boxes); b) whole pancreas extracted from early embryonic (E) to postnatal (P) stages; c) subfractions of pancreas from adult mice: acini, islets, β-cells and other islet cells (see Methods). We show the relative expression of Glis3 normalized to Gapdh, multiplied by a factor 1,000. The high degree of purity of the pancreas subfractions was validated by real-time quantitative PCR of pancreas genes, with expression ratio as follows: insulin I and II (islet/acini: 205:1), amylase (acini/islet: 278:1), insulin I and II (α-cells to other islet cells: 35:1), glucagon, polypeptide P and somatostatin (other islet cells to β-cells: 399:1, 86:1 and 17:1 respectively).

FIG. 5: Mutation in NDH1 patient

The sequence shown corresponds to GLIS3 RefSeq NM_{—}152629.2

FIG. 6: Human GLIS3 gene structure: alternative transcripts and predicted proteins

Left: GLIS3 transcripts. Black arraw: transcription start sites, as identified by 5′RACE experiments; grey arrows: likely additional 5′ ends of transcripts, observed in publicly available sequences (Genbank); Black squares: 3′ ends of transcripts, determined by RACE experiments and grey square: 3′ end of transcript, based on publicly available sequence (Genbank). Exons marked by a “k” were found to be alternatively spliced in some transcripts, based on our RT-PCR and sequencing experiments. Regions of exons encoding ZFDs are shown in crossed boxes. The coding regions in predicted transcripts are shawn in black. Exons are drawn to scale, with arbitrary inter-exons distances. The alternative 5′, 3′ or splice in exons is shown belaw the general structure, and their order corresponds to the code shown in Supplementary Table 3. 5′ ends in exons 13, 19, 20 and 24 were found in publicly available sequences and may be incomplete: exon 13: BC033899 (testis), exon 19: BX492880 (EST, flot shown), exon 20: BX493219 (neuronal), exon 24: AK096318 (EST, not shown). The internal 3′ end in exon 32 was found in a publicly available sequence (BC033899). 12 different start sites were found at various positions of exon 12. In this case, and in other cases of exons where multiple 5′ ends were identified, some of the internal sites may correspond to secondary substructures. Based on our exploration of tissue specificity (see Methods), exon 12, marked by “R” was specific to retina, and alternative exons 4 and 5 were specific to testis. Several other exons or exon variants also showed same tissue specificity: 7, 10, 11, 13, 14, 15, 16, 19, 20, 22, 23, 24, 26 and 30. Transcripts shown as full length were validated by Northern blot hybridization together with RT-PCR, 5′ to 3′ RT-PCR or RACE. The position of DNA probes used in Northern blots hybridizations is shown by black bars above the gene structure. The full structure of all transcripts was not determined, and only the 5′ end or 3′ end of some transcripts is shown. Alternative 5′ exons of same transcripts encoding the same predicted N-terminal protein region are shown in brackets. Additional 3′ ends of transcripts were found in Genbank clones or obtained in 3′ RACE experiments, which may correspond to genomic A-rich regions, and are not shown.

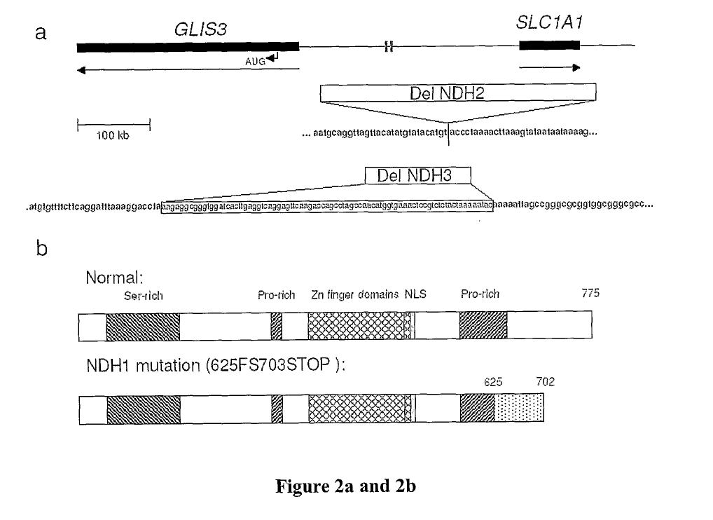

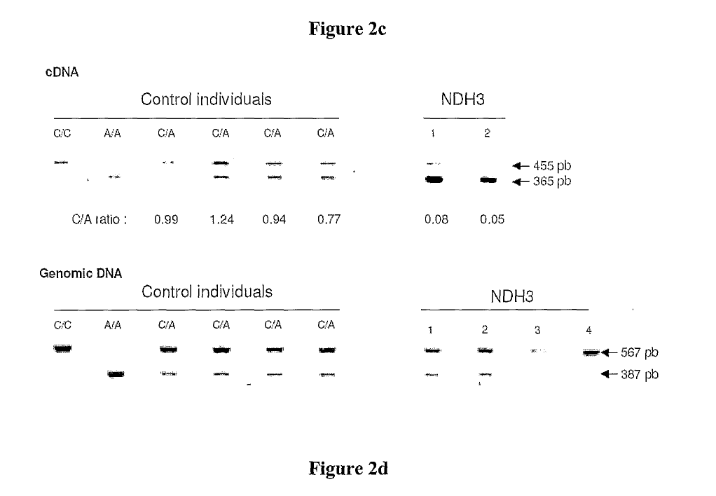

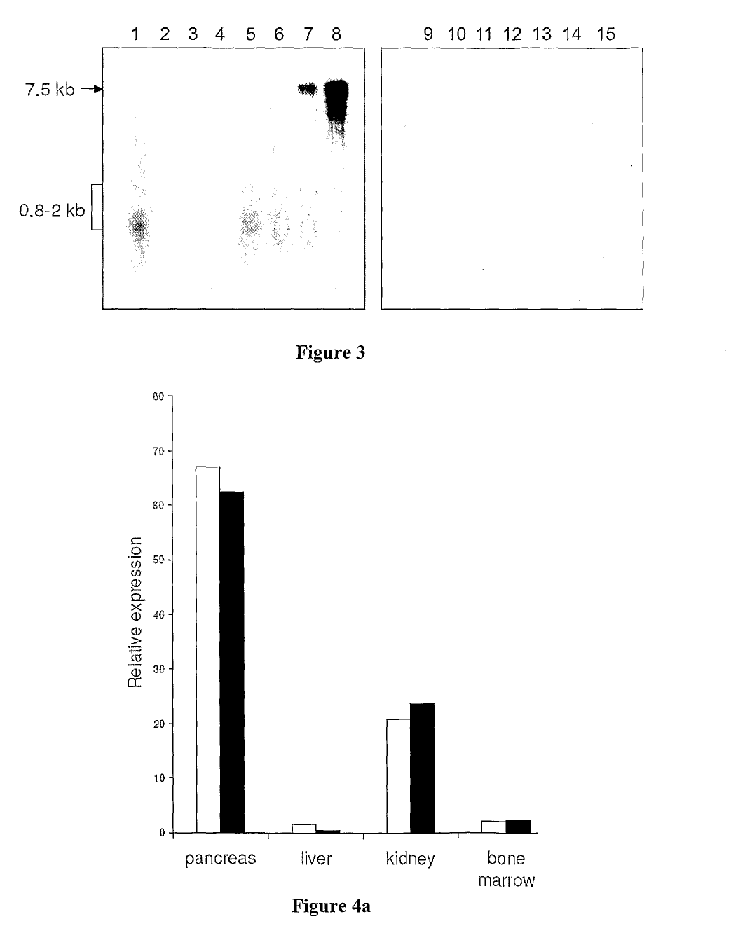

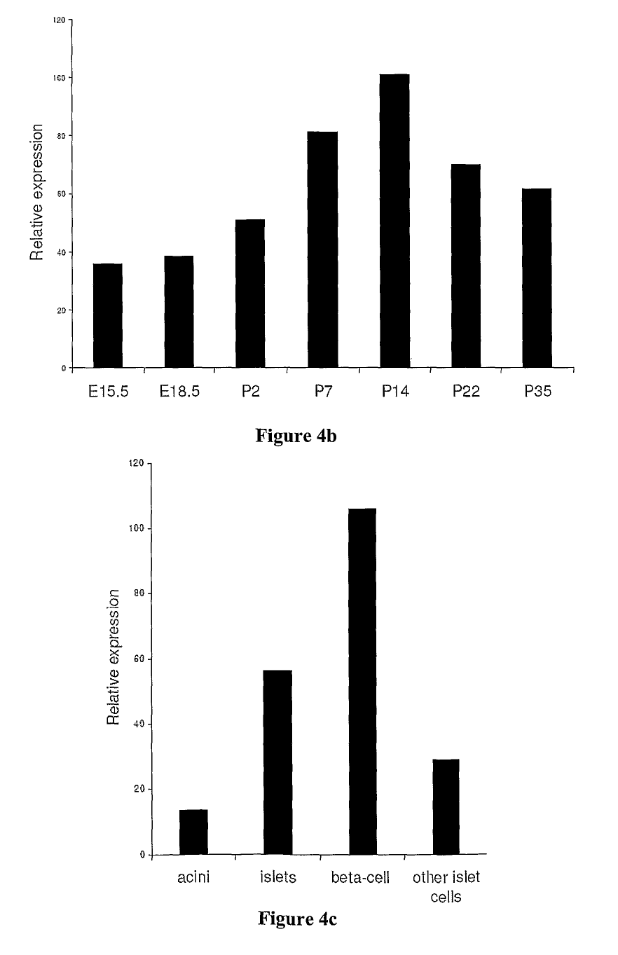

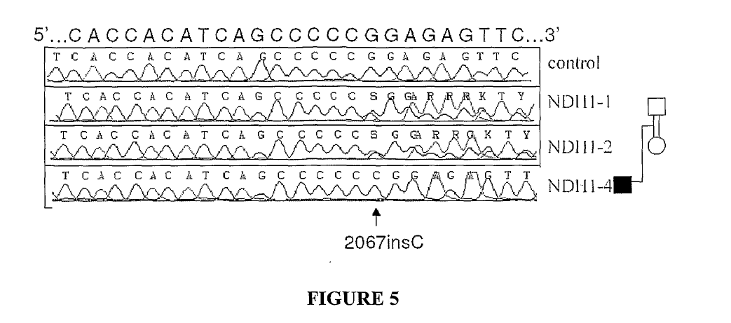

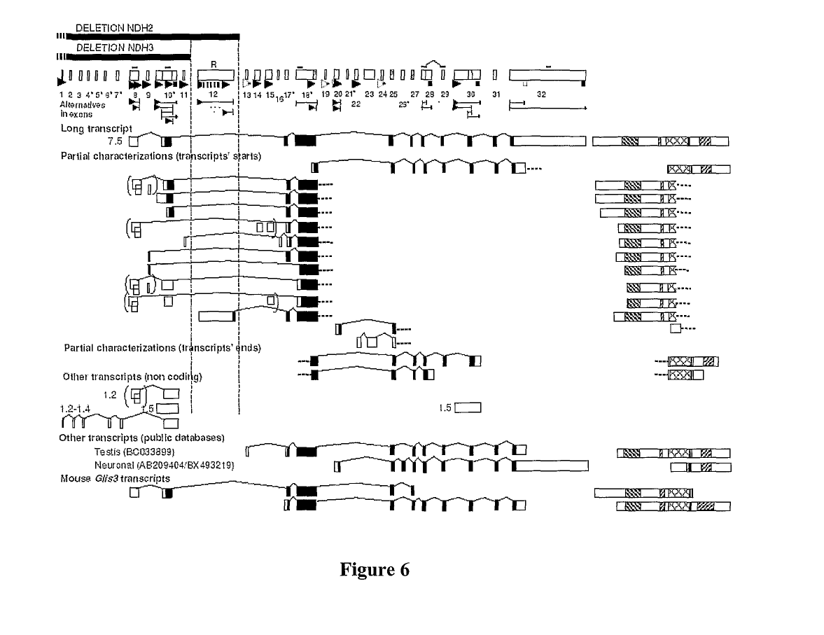

Right: Structure of the predicted proteins encoded by the different transcripts. Proteins with predicted ORF of 160 amino acids or less are not shown. Specific protein domains are represented as described in FIG. 2b legend.

Dotted lines indicate incomplete transcripts or predicted protein ends.

### EXAMPLE 1

### Identification of GLIS3 Mutations Responsible for Neonatal Diabetes and Loss of Functional Pancreatic Beta-Cells

### 1.1 Patients and Families

Family NDH1, a consanguineous family from Saudi Arabia, has been reported previously¹. A third child was born in this family, bearing the same clinical features, with the exception of the absence of cystic kidney disease, which may be related to the young age at death (10 days). All three patients died in young ages from pneumonia and respiratory failure (NDH1-3) or sepsis (NDH1-4 and -5). Blood samples were available from both parents, and NDH1-4. Family NDH2 was also a consanguineous family from Saudi Arabia. One child (NDH2-7) was affected with the same syndrome, with the exception that no liver and kidney defects were observed in this patient and that the facial dysmorphology was not as striking. The patient was alive (two years old). The patient, parents, and the four healthy siblings were available for study. Family NDH3 was a consanguineous French gypsy family, with two patients alive and presenting similar features, but without detectable liver and kidney defects and no glaucoma. All patients showed severe IUGR, and the diagnosis of neonatal diabetes was made at day 1 or 2.

The study was explained to family members, who signed an informed consent. NDH3 patients and their parents signed an additional consent for publication of their photographs in a scientific journal. The study protocol was approved by the local ethics committees (King Faisal Specialist Hospital, Jeddah, and Hôpital Saint-Vincent de Paul, Paris). Blood samples were collected, and DNA extraction performed using standard techniques. RNA extraction was performed on blood samples from patients NDH2-7 and his parents, and NDH3-3 and -4 and their parents, using PAXgene Blood RNA kit (QIAGEN).

TABLE 1
Clinical description of patients

| | | Country | | Neonatal | Congenital | Facial | Congenital | Liver | Cystic | Mental | Age at | |
|---|---|---|---|---|---|---|---|---|---|---|---|---|
| Family | Patient | of origin | IUGR | diabetes | hypothyroidism | dysmorphology | glaucoma | fibrosis | kidneys | retardation | death | |
| NDH1 | 3* | Saudi | + | + | + | + | + | + | + | NA | 16 | m |
| | | Arabia | | | | | | | | | | |
| NDH1 | 4 | Saudi | + | + | + | + | + | + | + | NA | 6 | m |
| | | Arabia | | | | | | | | | | |
| NDH1 | 5* | Saudi | + | + | + | + | + | + | − | NA | 10 | d |
| | | Arabia | | | | | | | | | | |

| | | | | | | | | | | | |
|---|---|---|---|---|---|---|---|---|---|---|---|
| NDH2 | 7 | Saudi | + | + | + | +/− | + | − | − | NA | alive (2 y) |
| | | Arabia | | | | | | | | | |
| NDH3 | 3 | France | + | + | + | + | − | − | − | mild | alive (22 y) |
| NDH3 | 4 | France | + | + | + | + | − | − | − | mild | alive (14 y) |

IUGR: intrauterine growth retardation; NA: not available, because of young age.
*DNA samples from these patients were not available.

### 1.2 Linkage Studies

Genome scan was performed by semi-automated fluorescent genotyping, using more than 400 microsatellite markers (Linkage Mapping Set 2, Applied Biosystems, with modifications), as described (http://www.cng.fr/fr/teams/microsatellite/index.html). Linkage analyses were performed using SIMWALK2 program, under a fully penetrant recessive model, assuming no heterogeneity.

### 1.3 Fine Mapping Studies

First, linkage confirmation and fine mapping were performed by genotyping all family members with known microsatellite markers. Additional microsatellite markers were developed by selecting 31 di-, tri-, tetra- and pentanucleotide repeat elements. 28 of these were found to be polymorphic. Precise definition of the deletion boundaries in NDH2 and NDH3 families was performed using 33 simple PCR assays, followed by PCR across the deletion, and sequencing specific PCR fragments obtained.

### 1.4 Mutation Screening by DNA Sequencing

The totality of the coding regions of the GLIS3 gene was sequenced in NDH1 patient and his parents on PCR fragments generated from genomic DNA using primers shown in Supplementary Table 2c. Sequence interpretation was performed using the Genalys software¹⁸.

SUPPLEMENTARY TABLE 2c
Sequences of primers used for mutation screening in NDH1 family

| | | | |
|---|---|---|---|
| | | PCR amplification | |
| Region | Map position^{a} | and sequencing primers (5′-3′) | Product |

| | | | | | |
|---|---|---|---|---|---|
| Amplified^{b} | Start (bp) | End (bp) | Forward | Reverse | Size (bp) |

| | | | | | |
|---|---|---|---|---|---|
| Exon8 | 4290317 | 4289708 | caggaagaaggcgaagtgtc | gctcattctcccctgctaca | 610 |
| Exon8 | 4289857 | 4289209 | gcagcaattagccagcatc | agtctcagtcagcgcaggtc | 649 |
| Exon9 | 4288781 | 4288278 | tcaccctgtggtttcctttc | ccgccaggtctggaactat | 506 |
| Exon10 | 4278787 | 4278207 | tgtagttgccgagagcactg | gttgttctgaggagccatcc | 581 |
| Exon12 | 4150429 | 4149313 | tggccctttctttcaatacg | taaggcattttcctggcatc | 1117 |
| Exon13 | 4142449 | 4141834 | agcctttggatgaaggctgt | cagcaggagagaacctttgg | 616 |
| Exon17 | 4118042 | 4115885 | ctgtctctaaagatgggtcctg | gagaacggaaaacacttgtgg | 378 |
| Exon18 | 4109027 | 4108423 | gcccaagaatgtgtgtttagc | aggaaatgcccgtagacctc | 605 |
| Exon18 | 4108828 | 4107933 | cctagtcctcggcactcatc | ctcctgctggtcgtacagg | 696 |
| Exon18 | 4108085 | 4107497 | catgcgcaccttcaccac | ctggcatctcctttccaaga | 589 |
| Exon20 | 4071848 | 4071082 | gtccctccaaatcacagtgg | agaatctgccccagaactgt | 587 |
| Exon24 | 3985525 | 3984987 | ccagaggtcagctaggttcc | aggatgtgtgggatttggag | 559 |
| Exon25 | 3927404 | 3928872 | tctgatacagaaagtggcatgt | ccccatctggccttttacc | 533 |
| Exon26 | 3923058 | 3922571 | accaggacacctctgcaact | ccaattgactgatgtgaaatgc | 486 |
| Exon27 | 3922884 | 3922141 | gggcatctctttgttccttg | gcaaaccatagccatatcagc | 544 |
| Exon28 | 3889089 | 3888528 | ctgaagtcaaatccgcctct | ccagctcaggaatcagaagg | 542 |
| Exon29 | 3889841 | 3889235 | tggcagcacttaggagacag | cccgtggaaaaatgaaacag | 607 |
| Exon30 | 3848282 | 3845890 | atagccagaatgggaggtga | tgaaagcctaagcctggtgt | 393 |
| Exon31 | 3819888 | 3819044 | cttcagcccaacagtgcat | gaggaatcaggtcagggagac | 623 |
| Exon32 | 3818584 | 3817975 | tcgctggaaacagagactca | gggttgacagccaggaagta | 590 |
| Exon32 | 3818453 | 3817838 | catggggcaggagttactga | cagcaagggtgaggattagg | 616 |
| Exon32 | 3818082 | 3817442 | tggactatgtgaccaggaga | actgttggtggggaggt | 621 |

### 1.5 Expression Studies

### Northern Blots Hybridization

We used commercially available Northern blots (BD Biosciences, Clontech) for evaluation of GLIS3 expression in normal adult tissues. As specified by the manufacturer, the RNA amount loaded in each lane was adjusted to obtain similar hybridization signal using a β-actin probe. Probes were generated by PCR amplification on cDNA or genomic DNA, confirmed by sequencing and labelled with ³²P-dCTP using Megaprime DNA labelling system (Amersham), and hybridization was performed according to manufacturer's recommendations, with a final stringent wash in 0.1×SSC, 0.1% SDS buffer at 60° C.

### 1.6 RT-PCR Expression Studies in Normal Tissues and in Patients and their Family

RT-PCR was performed on commercially available RNA from normal adult tissues (pancreas, kidney, thyroid, liver, retina, heart, skeletal muscle, placenta, lung, brain, testis, BD Biosciences, Clontech) on poly A+ RNA (all tissues, except retina) or total RNA (retina), using M-MLV Reverse Transcriptase (invitrogene), following manufacturer's instructions. RT-PCR was performed on whole blood RNA from NDH2 and NDH3 families, using the same protocol, with the exception that nested or double PCR amplification of cDNA was used in some instances, as follows. The first round PCR mixture was diluted 50 times and served as a template for the second round PCR (nested-PCR using internal primers, or double-PCR using the same primers), consisting of 20 cycles of PCR. Control RT-PCR was performed using primers located in the ubiquitously expressed PCBD1 gene (6-pyruvoyl-tetrahydropterin synthase/dimerization cofactor of hepatocyte nuclear factor 1 alpha). Sequence of primers used for PCR and nested PCR in NDH2 and NDH3 families are provided in Supplementary Table 2d below.

SUPPLEMENTARY TABLE 2d
PCR assays used for RT-PCR expression studies in NDH2 and NDH3 families

| | Primers used for 1st PCR (5′-3′) | | Primers used for nested PCR (5′-3′) | | Product |
|---|---|---|---|---|---|
| Gene | Forward | Reverse | Forward | Reverse | size (bp) |

| | | | | | |
|---|---|---|---|---|---|
| GLIS3 | ctccatccagacctgctcac | gctgtgagtggaggtaactgg | cctagagatgctgctgctga | cctggagaagggtgactgac | 158 |
| (nested | | | | | |
| PCR) | | | | | |
| SLC1A1 | ccagaaagtgggtgaaattg | atgtaaaggcccagcttgc | cggtggatgccatgttagat | gatcttcccagcaatcagga | 398 |
| (nested | | | | | |
| PCR) | | | | | |
| PCBD1 | ctggaaggccgtgatgccatc | tgtggaccttgttgtacacgt | | | 142 |
| | ttc | | | | |

SUPPLEMENTARY TABLE 2e

RT-PCR assay for quantitative expression study of GLIS3 in NDH3 family, and
PCR assay for genomic genotyping of GLIS3 SNP rs6415788

| | | |
|---|---|---|
| | | PCR fragment sizes |
| Primers (5′-3′) | Product size (bp) | after Styl digestion |

| | | | | |
|---|---|---|---|---|
| Forward | Reverse | (before digestion) | allele C (uncut) | Allele A (cut) |

| | | | | | |
|---|---|---|---|---|---|
| cDNA | ggcctgttcaagaccgaac | gccttctcgcaaccttca | 455 | 455 | 365, 90 |
| genomic DNA | cctagtcctcggcactcatc | ctcctgctggtcgtacagg | 696 | 567 (−129) | 387, 180 (+129) |

### 1.7 Allele-Specific Expression Quantification

RT-PCR was performed on blood cDNA from parents heterozygous for the deleted GLIS3 allele and heterozygous for the exonic SNP rs6415788, located in RefSeq exon 3 (exon 18.1 in the final structure) and from control individuals heterozygous or homozygous for each allele of this SNP, using two rounds of PCR with primers located in RefSeq exons 3 and 4 (exons 18.1 and 25 in the final structure, Supplementary Table 2e on line). PCR was also performed on genomic DNA from the same individuals, using distinct primers. After restriction enzyme digest with Styl at 37° C. for 16 h, which discriminates the two alleles, the PCR products were separated on a 2% agarose gel and the intensity of bands specific for each allele was quantified using an AlphaImager (Alpha Innotech Corporation).

The structure of GLIS3 gene was established using the combination of three methods: 5′- and 3′-Rapid Amplification of cDNA ends (RACE) experiments in pancreas, kidney and retina (as a representative tissue for eye), inter-exon RT-PCRs and Northern blot hybridizations. 5′- and 3′-ends were determined using 5′- and 3′-RACE on polyA+ RNA from kidney and pancreas or total RNA from retina (BD Biosciences Clontech) using the BD SMART™ RACE cDNA Amplification Kit (BD Biosciences Clontech), following the manufacturer's instructions; we used primers located in all exons referenced in AceView or located in referenced mRNAs or spliced transcripts, as well as novel exons that we identified during the course of this study. The potential 3′-ends identified were critically reviewed and those that may correspond to genomic polyA regions or internal polyA regions in transcripts were excluded. Primers sequences used for RACE experiments are provided in Supplementary Table 2f.

SUPPLEMENTARY TABLE 2f
Primers used to generate a GLIS3 probe for
Human Northern blot hybridization

| | | PCR product |
|---|---|---|
| Forward (5′-3′) | Reverse (5′-3′) | size (bp) |
| GGCCTGTTCAAGACCGAAC | GCCTTCTCGCAACCTTCA | 275 |

We used RT-PCR and long range RT-PCR (HotStarTaq, Qiagen) between exons, in cDNAs from five tissues (pancreas, thyroid, liver, kidney and retina) followed by sequencing of the PCR products, in order to identify and validate alternative transcripts. Transcripts that were not expressed in all five tissues were then tested in the extended panel of tissues to explore tissue-specific expression. All the information generated using these strategies, and publicly available data (NCBI clones sequences and map information) were merged into a combined database and graphic display using a locally developed database management program (C.C., unpublished). Predicted transcripts were compared to transcripts sizes determined by Northern blot hybridization of exon-specific probes. A comprehensive GLIS3 gene structure, with the coding prediction of transcripts is shown in FIG. 6 and the detailed intron-exon structure in Supplementary Table 3.

SUPPLEMENTARY TABLE 3
Exon-intron structure of the human GLIS3 gene

| | Genomic position (NT_008413.16) | | |
|---|---|---|---|
| Exons | Start (bp) | End (bp) | Exon size (bp) |
| 1 (5′) | 4338392 | 4338353 | 40 |
| 2 | 4338336 | 4338232 | 105 |
| 3 | 4337183 | 4337081 | 103 |
| 4 | 4319891 | 4319776 | 116 |
| 5 | 4301241 | 4301122 | 120 |
| 6 | 4300528 | 4300401 | 128 |
| 7 | 4298968 | 4298777 | 192 |
| 8.1 (5′) | 4289916 | 4289421 | 496 |
| 8.2 (5′) | 4289594 | 4289421 | 174 |
| 9 (5′) | 4288496 | 4288384 | 113 |
| 10.1 (5′, 3′) | 4276996 | 4275539 | 1458 |
| 10.2 (5′) | 4276996 | 4276038 | 959 |
| 10.3 | 4276523 | 4276038 | 486 |
| 10.4 (3′) | 4276523 | 4275539 | 985 |
| 10.5 (5′) | 4276335 | 4276038 | 298 |
| 11 (5′) | 4270321 | 4270222 | 100 |
| 12.1 (5′) | 4151383 | 4149405 | 1979 |
| 12.2 (5′) | 4150823 | 4149405 | 1419 |
| 12.3 (5′) | 4150697 | 4149405 | 1293 |
| 12.4 (5′) | 4150362 | 4149405 | 958 |
| 12.5 (5′) | 4150350 | 4149405 | 946 |
| 12.6 (5′) | 4150250 | 4149405 | 846 |
| 12.7 (5′) | 4150151 | 4149405 | 747 |
| 12.8 (5′) | 4150139 | 4149405 | 735 |
| 12.9 (5′) | 4150016 | 4149405 | 612 |
| 12.10 (5′) | 4149963 | 4149405 | 559 |
| 12.11 (5′) | 4149940 | 4149405 | 536 |
| 12.12 (5′) | 4149881 | 4149405 | 477 |
| 13 (5′)* | 4142183 | 4142067 | 117 |
| 14 (5′) | 4137644 | 4137297 | 348 |
| 15 (5′) | 4135193 | 4134839 | 355 |
| 16 | 4133087 | 4133033 | 55 |
| 17 | 4115941 | 4115734 | 208 |
| 18.1 | 4108881 | 4107768 | 1114 |
| 18.2 (5′) | 4108134 | 4107768 | 367 |
| 19 (5′)* | 4098886 | 4098853 | 34 |
| 20.1 (5′)* | 4071361 | 4071228 | 134 |
| 20.2 (5′) | 4071301 | 4071228 | 74 |
| 21 | 4063394 | 4063320 | 75 |
| 22 (5′) | 4025998 | 4025838 | 161 |
| 23 | 4025042 | 4024527 | 516 |
| 24 (5′)* | 3965199 | 3965134 | 66 |
| 25 | 3927189 | 3927028 | 162 |
| 26 | 3922918 | 3922795 | 124 |
| 27 | 3922470 | 3922360 | 111 |
| 28.1 | 3888835 | 3888691 | 145 |
| 28.2 (3′) | 3888835 | 3888254 | 582 |
| 29 | 3869595 | 3869427 | 169 |
| 30.1 (5′, 3′) | 3846915 | 3845437 | 1479 |
| 30.2 (5′) | 3846915 | 3846009 | 907 |
| 30.3 | 3846184 | 3846009 | 176 |
| 30.4 (3′) | 3846184 | 3845437 | 748 |
| 31 | 3819492 | 3819310 | 183 |
| 32.1 (3′)** | 3818408 | 3817677 | 732 |
| 32.2 (3′) | 3818408 | 3814127 | 4282 |

Notes:
Exons with variable 5′ or 3′ ends (or splice) are shown with the same first number, and different extensions: the presence of a 5′ end (to the splice) or 3′ end (from the splice), both 5′ and 3′ ends, or both ends spliced (no indication) is shown in parentheses.
5′ exons marked by a * correspond to putative 5′ exons, based on clones available in Genbank, and may be incomplete.
3′ exon marked by a ** was observed in clone BC033899, and was not identified in our RACE studies.
This structure is based on our RACE studies in kidney, pancreas and retina, and RT-PCR in multiple tissues, completed by information from Genbank clones, as described in Methods.

### 1.8 Expression Studies in Mouse Tissues, Pancreas Subfractions and During Mouse Development

RNA was isolated from whole mouse tissues including pancreas, liver, kidney and bone marrow at postnatal ages 22 and 35 days, and whole mouse pancreata at different developmental stages from early embryonic (E15.5) to adult mice. Pancreas subfractions were generated as follows: Pancreata from adult (P35) transgenic mice which express green fluorescent protein under the control of mouse insulin I gene promoter (MIP-GFP mice)¹⁹) were isolated and subjected to collagenase (1 mg/ml) digestion for 20 minutes. Collagenase digested pancreata were fractionated by histopaque centrifugation to isolate islets and acinar cells. Islets and acinar cell clusters were then purified by handpicking under a stereomicroscope. A portion of the islets was further disassociated by trypsin (0.125% in PBS) digestion to release single cells. Disassociated islets cells were fractionated by FACS sorting based on GFP fluorescence of the MIP-GFP transgene, specific for β-cells, resulting in GFP+ (β-cells) and GFP- (other islet cells). RNA was extracted for all tissues and isolated cells using RNeasy Mini Kit (Qiagen, Inc.). cDNA was prepared by standard reverse transcription of total RNA. Glis3 expression was quantified by real time quantitative PCR on an ABI 7000 (Applied Biosystems, Inc.), using Glis3 specific primers and Gapdh primers for normalization, following manufacturer's instruction. We report the mean Glis3 expression value (relative to Gapdh) of three independent experiments performed using primer pairs located in mouse Glis3 gene in exons 28 and 29 (one pair) and in exons 8 and 10 (two pairs). We confirmed by hybridization of a mouse Northern blot (not shown) that exons 8, 10, 28 and 29 belong to the major (7.5 kb) mouse transcript, like in human.

### URL

NCBI AceView: http://www.ncbi.nlm.nih.gov/IEB/Research/Acembly/

### Genbank Accession Numbers

Human GLIS3 cDNA: NM_{—}152629.2 (RefSeq), Human SLC1A1 cDNA: NM_{—}004170.4 (RefSeq), Human genomic DNA of GLIS3 chromosome region: NT_{—}008413.16. Human GLIS3 protein: NP_{—}689842.2 (RefSeq). Sequences generated in this study have been submitted to GenBank under the access numbers DQ438877 to DQ438907.

### 2. Results

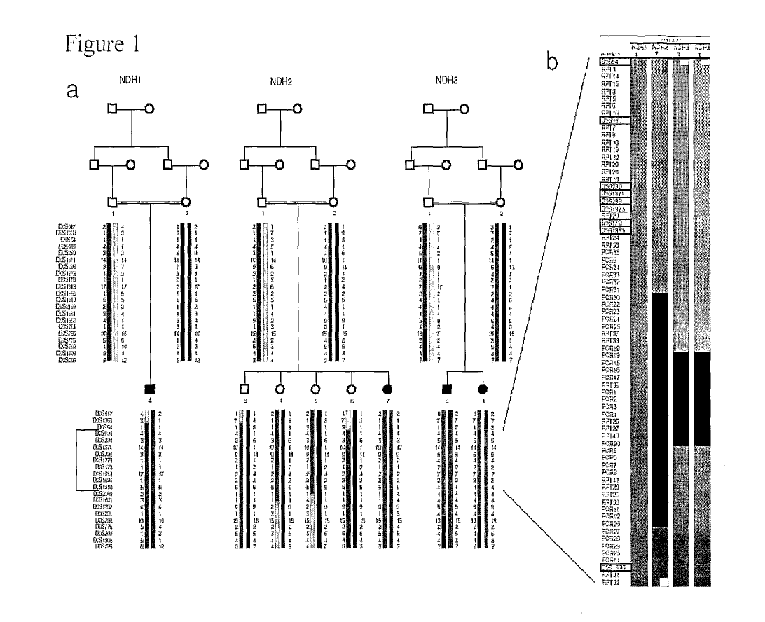

We performed a genome-wide linkage scan on all family members available, and obtained suggestive evidence of linkage, under a fully penetrant recessive model, to a single chromosome region on chromosome 9p, which we then confirmed to a 7.3 cM region (LOD=4.67) using additional markers (FIG. 1a). Using 31 newly developed microsatellite and 33 simple PCRs assays, we identified two distinct deletions in families NDH2 and NDH3 (FIG. 1b), and we determined the deletion breakpoints by PCR across the deletion and sequencing (FIG. 2a). NDH2 patient carried a homozygous 426 kb deletion, which contains the gene encoding the neuronal/epithelial high affinity glutamate transporter (SLC1A1) and part of the 5′UTR of the gene encoding GLI similar 3 (GLIS3), as defined in NCBI AceView prediction (http://www.ncbi.nih.gov/IEB/Research/Acembly/). NDH3 patients carried a homozygous 149 kb deletion, which only contained part of the 5′UTR region of GLIS3. The region common to both deletions mapped 134 kb 5′ to the known start codon of GLIS3 (NCBI RefSeq). GLIS3 is a recently identified transcription factor containing five Krüppel-like zinc finger domains, sharing high homology with GLI zinc finger proteins, which has been postulated to play a critical role in the regulation of a variety of cellular processes during development². Based on its presumed function, the presence of GLIS3 exons in the region common to NDH2 and NDH3 deletions, and the absence of SLC1A1 involvement in NDH3 patients, GLIS3 appeared as the likely responsible gene for this syndrome. We sequenced all the coding exons of GLIS3 in family NDH1 and identified a homozygous insertion (2067insC; FIG. 5) leading to a frameshift and a truncated protein (625FS703STOP), in which the C-terminal proline-rich domain is altered (FIG. 2b). Deletions affecting the region C-terminal to Thr713 of mouse Glis3 (Thr708 in human) have been shown to abolish almost completely GLIS3 activity in vitro². The 625FS703STOP mutation is therefore expected to result in a non-functional protein. This mutation was not found in 175 normal Caucasian individuals. No mutation was identified in the SLC1A1 gene in NDH1 patient (data not shown). These results support that the frameshift mutation (NDH1) and deletions (NDH2 and NDH3) affecting GLIS3 are responsible for this syndrome.

The coding sequence of GLIS3 (according to RefSeq) was intact in NDH2 and NDH3 patients, so that some expression and coding capacity may be retained in these patients. Using RT-PCR, we explored GLIS3 expression on RNA extracted from whole blood from NDH2 and NDH3 patients and parents (FIG. 2c). After simple RT-PCR, no GLIS3 expression was detected in patients carrying homozygous deletions, while it was expressed in heterozygous parents and normal control individual; however, after nested PCR, some expression was detected in these patients. We quantified the relative expression of GLIS3 from the normal and “deleted” alleles in NDH3 parents, who were heterozygous for an exonic GLIS3 SNP. The expression of the “deleted” allele was reduced to 8 and 5% (average: 6%) that of normal, while both alleles showed similar expression levels in control individuals (mean expression ratio=0.98, FIG. 2d). In contrast, SLC1A1 was normally expressed in NDH3 patients (FIG. 2c).

The complete deletion of SLC1A1 in NDH2 patient was not associated with additional clinical features compared to NDH1 patients, who carry an intact SLC1A1 gene. SLC1A1 is principally expressed in neurons, kidney and small intestine³. Slc1a1 knockout mice have dicarboxylic aminoaciduria, but are phenotypically normal⁴. Aminoaciduria patterns were normal in NDH2 and NDH3 patients, suggesting that SLC1A1 deletion has no or undetectable pathological consequences in human. These observations rule out SLC1A1 deletion as the cause of the phenotypic variability between families.

Because of the extremely reduced expression of GLIS3 transcripts in blood in NDH2 and NDH3 patients, despite the presence of the coding and 5′ exons as defined in RefSeq, we hypothesized that the distant region deleted in NDH2 and NDH3 patients contains sequences that are critical for GLIS3 expression. In order to explore this hypothesis, we established the structure of the GLIS3 gene, and tested its expression in relevant tissues.

Using a cDNA probe encompassing two coding exons present in all reported GLIS3 transcripts, we detected multiple transcripts in Northern blot hybridization (FIG. 3). These transcripts are expressed in most tissues, with variability in size and level of expression. The sizes of the major transcripts were approximately 7.5 kb and smaller (0.8-2.0 kb). Additional bands of lower intensity were also visible. The ratio of the 7.5 kb to smaller transcripts was highly variable depending on the tissue. The smaller transcripts showed strong expression in liver, kidney, heart and skeletal muscle, and the 7.5 kb in pancreas, thyroid and kidney, with an extreme bias towards expression of the 7.5 kb transcript in pancreas and thyroid. We established the structure of the GLIS3 gene (Supplementary Table 3), and characterized the alternative transcripts and predicted protein products (FIG. 6). We identified 25 putative transcription start sites located in twelve exons, and four transcription termination sites, located in four exons; four additional 5′ ends (including three additional exons) and one 3′ end of clones were present in sequence databases (GenBank). Some of the transcription start sites appeared to be tissue-specific. In particular, the 5′ ends located in exon 12 were found only in retina. The finding of multiple 5′ and 3′ ends and alternatively spliced exons is consistent with the observation of multiple transcripts, with qualitative and quantitative variation in expression depending on the tissues.

The predicted translation products of these transcripts encode variants of the GLIS3 protein, most containing the five intact zinc finger domains (ZFDs), with variations in the N- and C-terminal regions (FIG. 6), which may affect the function of the encoded proteins, as previously shown by Kim et al. ². In particular, the predicted protein encoded by the 7.5 kb transcript contains 492 additional N-terminal amino-acids compared to the one starting in exon 18 (930 vs. 438 amino-acids). Some transcripts have no or limited coding capacity, and may have regulatory function.

The seven most 5′ start sites and one 3′ end are located in the region common to both deletions in NDH2 and NDH3 patients, and the multiple start sites in exon 12 are located in a region specific to the NDH3 deletion. Therefore, some of these transcripts, including the 7.5 kb variant, will be lacking in patients carrying homozygous deletions, while others, including the one starting in exon 18, will be present, and the retina-specific transcripts starting in exon 12 will be missing in NDH2 but present in NDH3 patients. The pancreas and the thyroid, whose main transcript is the 7.5 kb variant, but not the liver and the kidney, are affected in NDH2 and NDH3 patients, which suggests that this transcript is required for the development or function of the pancreas and the thyroid, while other GLIS3 transcripts are sufficient in the other organs. Similarly, the retina-specific transcripts starting in exon 12 encode a long N-terminal protein, which is predicted to be present in NDH3 patients, who do not have glaucoma, but missing in NDH2 patient, who has congenital glaucoma, suggesting a critical role for these transcripts in eye development. Alternatively, the more severe clinical phenotype in NDH1 patients may be explained in part by the nature of the defective GLIS3 products encoded, and some of the clinical variability may also depend on other factors, such as modifier genes and environmental factors.

In order to gain some insight into the mechanisms involved in NDH syndrome, we studied the expression of Glis3 transcripts in mouse pancreas during development and in adult pancreas subfractions. Similarly to human, Glis3 is expressed at high level in mouse pancreas compared to other tissues (FIG. 4a). Glis3 is expressed as a major transcript of 7.5 kb in mouse (Kim et al. ², and data not shown). During mouse development, Glis3 is expressed at embryonic stages as early as E15.5, at the time of pancreatic β-cells neogenesis, and its expression increases postnatally (FIG. 4b). We quantified Glis3 transcripts in microdissected islets and acini, in purified β-cells and in other endocrine cells. As shown in FIG. 4c, while Glis3 is expressed in all pancreatic tissues, its expression is increased in β-cells compared to other islet cells and to acini (expression ratio: 8:2:1). Hence, Glis3 is likely to play a critical role during pancreatic development, and particularly that of β-cells.

On the basis of its strong expression in pancreatic β-cells, its expression in pancreas from early stages of β-cells neogenesis, its lower expression in the exocrine pancreas, and the absence of exocrine pancreas dysfunction in patients, the neonatal diabetes observed in this syndrome is likely to result from pancreatic β-cell failure, rather than a general defect affecting both the exocrine and the endocrine pancreas, as observed in neonatal diabetes with pancreas agenesis^{5,6}, or a primary defect of the exocrine pancreas associated with diabetes⁷. The complex pattern of GLIS3 transcripts and encoded proteins, their variability between tissues, and the fact that GLIS3 may function both has a repressor or activator of transcription², may contribute to the fine-tuning required in the development and function of several organs.

Congenital glaucoma has been reported in some cases of unbalanced translocations with monosomy of the distal end of chromosome 9p⁸⁻¹⁰, overlapping with the GLIS3 locus. Although no gene for congenital hypothyroidism and congenital glaucoma has been mapped to this region to date^{10,11}, our results warrant further investigations of GLIS3 in these congenital disorders.

This is the first report of a disease associated with the family of GLIS genes, which play a role during embryonic development^{2, 12, 13}. Homozygous mutations in other transcription factors that play a role in pancreas and β-cell development, IPF1 and PTF1A, have been shown to result in neonatal diabetes in human^{5,6}, and the lack of Tcf2 (Hnf1 beta) in pancreas agenesis in the mouse¹⁴. Mutations in GLI3, a member of the GLI Kruppel-like zinc finger proteins, are responsible for various birth defects syndromes¹⁵, and GLI proteins act as downstream regulators of transcription in the hedgehog signaling pathway, which is involved in various development and cell differentiation processes^{15, 16}. As this pathway is essential for the proper development and function of the pancreas^{16, 17}, it is tempting to speculate that GLIS3, like GLI proteins, may be part of this pathway, hence contributing to the development and function of the pancreas, and other organs.

### REFERENCES

1. Taha, D., Barbar, M., Kanaan, H. & Williamson Balfe, J. Neonatal diabetes mellitus, congenital hypothyroidism, hepatic fibrosis, polycystic kidneys, and congenital glaucoma: a new autosomal recessive syndrome? Am J Med Genet A 122, 269-273 (2003).
2. Kim, Y. S., Nakanishi, G., Lewandoski, M. & Jetten, A. M. GLIS3, a novel member of the GLIS subfamily of Kruppel-like zinc finger proteins with repressor and activation functions. Nucleic Acids Res 31, 5513-5525 (2003).
3. Kanai, Y. & Hediger, M. A. Primary structure and functional characterization of a high-affinity glutamate transporter. Nature 360, 467-471 (1992).
4. Peghini, P., Janzen, J. & Stoffel, W. Glutamate transporter EAAC-1-deficient mice develop dicarboxylic aminoaciduria and behavioral abnormalities but no neurodegeneration. Embo J 16, 3822-3832 (1997).
5. Stoffers, D. A., Zinkin, N. T., Stanojevic, V., Clarke, W. L. & Habener, J. F. Pancreatic agenesis attributable to a single nucleotide deletion in the human IPF1 gene coding sequence. Nat Genet 15, 106-110 (1997).
6. Sellick, G. S. et al. Mutations in PTF1A cause pancreatic and cerebellar agenesis. Nat Genet 36, 1301-1305 (2004).
7. Raeder, H. et al. Mutations in the CEL VNTR cause a syndrome of diabetes and pancreatic exocrine dysfunction. Nat Genet. 38, 54-62 (2006).
8. Katsushima, H., Kii, T., Soma, K., Ohyanagi, K. & Niikawa, N. Primary congenital glaucoma in a patient with trisomy 2q (q33- - - - qter) and monosomy 9p(p24- - - - pter). Case report. Arch Ophthalmol 105, 323-324 (1987).
9. Verbraak, F. D. et al. Congenital glaucoma in a child with partial 1q duplication and 9p deletion. Ophthalmic Paediatr Genet 13, 165-170 (1992).
10. Cohn, A. C. et al. Chromosomal abnormalities and glaucoma: a case of congenital glaucoma with trisomy 8q22-qter/monosomy 9p23-pter. Ophthalmic Genet 26, 45-53 (2005).
11. Park, S. M. & Chatterjee, V. K. Genetics of congenital hypothyroidism. J Med Genet 42, 379-389 (2005).
12. Kim, Y. S. et al. Identification of Glis1, a novel Gli-related, Kruppel-like zinc finger protein containing transactivation and repressor functions. J Biol Chem 277, 30901-30913 (2002).
13. Zhang, F. et al. Characterization of Glis2, a novel gene encoding a Gli-related, Kruppel-like transcription factor with transactivation and repressor functions. Roles in kidney development and neurogenesis. J Biol Chem 277, 10139-10149 (2002).
14. Haumaitre, C. et al. Lack of TCF2/vHNF1 in mice leads to pancreas agenesis. Proc Natl Acad Sci USA 102, 1490-1495 (2005).
15. Villavicencio, E. H., Walterhouse, D. O. & Iannaccone, P. M. The sonic hedgehog-patched-gli pathway in human development and disease. Am J Hum Genet 67, 1047-1054 (2000).
16. Lees, C., Howie, S., Sartor, R. B. & Satsangi, J. The hedgehog signalling pathway in the gastrointestinal tract: implications for development, homeostasis, and disease. Gastroenterology 129, 1696-1710 (2005).
17. Hebrok, M. Hedgehog signaling in pancreas development. Mech Dev 120, 45-57 (2003).
18. Takahashi, M., Matsuda, F., Margetic, N. & Lathrop, M. Automated identification of single nucleotide polymorphisms from sequencing data. J Bioinform Comput Biol 1, 253-265 (2003).
19. Hara, M. et al. Transgenic mice with green fluorescent protein-labeled pancreatic beta-cells. Am J Physiol Endocrinol Metab. 284, E177-183 (2003).

1. A method of diagnosis of neonatal diabetes, congenital hypothyroidism and congenital glaucoma, comprising detecting in the sample of a patient or in a prenatal sample the presence or the absence of a mutation in the GLIS3 gene selected for stop mutations, deletions and insertions resulting in frame-shift, wherein the presence of said mutation is indicative of neonatal diabetes, congenital hypothyroidism and congenital glaucoma.

2. The method according to claim 1 comprising the use of probes to detect the following mutations:
deletion and insertion as shown in SEQ ID No 1 (Position of the deletion on the genomic sequence of reference (NT_{—}008413.16):
. . . 4249914[DEL4249915-4398572; substituted by seq [symbol] I [symbol] ]4398573 . . .
deletion and insertion as shown in SEQ ID No 2 (Position of the deletion on the contig of reference (NT_{—}008413.16):
. . . 4176076[DEL:4176077-4601776]4601777 . . .
Mutation BCO33899-insC2067-2068 (cDNA) or AL137071-insC92318-92319 (genomic): Insertion of a [symbol] C [symbol] between nucleotides 2067 and 2068 of the GLIS3 gene, sequence cDNA Genbank: BCO33899, or between nucleotides 92318-92319 (genomic) leading to a frameshift beginning at position 625 with an altered sequence till position 702 (SEQ ID No 4), and a STOP in position 703 (625FS703STOP).

3. The method according to claim 2 wherein the probes comprise a sequence displaying from 10 to 30 consecutive nucleotides of sequences SEQ ID No 1, 2, 5, 6 7, 8 and 9 or of a complementary sequence thereof, or of a sequence encoding SEQ ID No 4 to detect the presence of the inserted cytosine at position 2068.

4. The method according to claim 3 wherein the probes are labeled with fluorescent or bioluminescent molecules.

5. The method according to claim 1 comprising a RT-PCR reaction on the RNA extracted from the sample.

6. Primer or probe consisting of a sequence displaying from 10 to 30 consecutive nucleotides of sequences SEQ ID No 1, 2, 5, 6 7, 8 and 9 or of a complementary sequence thereof, or of a sequence encoding SEQ ID No 4 to detect the presence of the inserted cytosine at position 2068.

7. Use of GLIS3 protein (SEQ ID No 3) to produce functional pancreatic beta-cells.

8. A method for preparing functional pancreatic beta-cells by culturing human multipotent or pluripotent cells, such as embryonic stem cells (ES-cells) or human somatic stem cells in a culture medium comprising an effective amount of GLIS3 to induce said cells differenciation into functional pancreatic beta-cells producing insulin.

9. A culture medium comprising an effective amount of GLIS3.

10. A cell culture comprising human multipotent or pluripotent cells, such as embryonic stem cells (ES-cells) or human somatic stem cells in a culture media comprising GLIS3.

11. A method of treatment of type 1 diabetes, type 2 diabetes, other forms of diabetes, hypothyroidism and glaucoma comprising administering an effective amount of GLIS3 to a patient in need of such treatment.

12. A pharmaceutical composition comprising GLIS3 as active ingredient.

13. A gene therapy vector expressing GLIS3.
